(19) United States
(12) Patent Application Publication
(10) Pub. No.: US 2003/0045804 A1
(43) Pub. Date: Mar. 6, 2003

(54) Method and apparatus for generating electrocardiogram precordial leads using a precordial central terminal

(75) Inventor: Donald E. Brodnick, Cedarburg, WI (US)

Correspondence Address: MICHAEL BEST & FRIEDRICH, LLP, 100 E WISCONSIN AVENUE, MILWAUKEE, WI, 53202, US

(73) Assignee: G.E. Medical Systems Information Technologies, Milwaukee, WI

(21) Appl. No.: 09/682,421

(22) Filed: Aug. 31, 2001

(51) Int. Cl. A61B 5/0428

(52) U.S. Cl. 600/509

(57) ABSTRACT

A method and apparatus for generating a plurality of ECG precordial leads using a precordial central terminal derived from a plurality of electrodes for attachment around the circumference of a patient's upper torso. The apparatus is a device including a plurality of electrodes, an acquisition module including a signal processor coupled to the plurality of electrodes for acquiring electrical signals from the electrodes and for generating a plurality of ECG precordial leads from the acquired electrical signals. According to the method of the invention, an approximation of the electrical potential near the center of the patient's heart (i.e., a precordial central terminal) is generated based on the signals acquired from the plurality of electrodes. The precordial central terminal signal is subtracted from the signal acquired from each electrode attached to the patient's chest in order to generate the precordial leads.

### BACKGROUND OF INVENTION

The invention relates to a method and apparatus for generating a plurality of electrocardiogram (ECG) precordial leads using a precordial central terminal derived from signals from a plurality of electrodes attached to a patient's upper torso.

It is commonly known that ten electrodes and ten leadwires are needed to record what is referred to as a standard, twelve-lead ECG (hereinafter standard ECG). As shown in FIG. 1, for a standard ECG, one electrode is attached to each of the patient's limbs. Specifically, one electrode is attached to the patient's right arm (eRA), left arm (eLA), left leg (eLL), and right leg (eG). The electrode attached to the patient's right leg (eG) acts as an electrical ground. Additionally, six electrodes (eV1, eV2, eV3, eV4, eV5, and eV6) are attached to the patient's chest in the area over the heart. The ten electrodes are connected via several resistor networks to enough amplifiers to record twelve leads (i.e. electrical signals) of ECG data.

The twelve leads of ECG data are split into two groups: the frontal plane and the horizontal plane. If a straight line were drawn from the heart to each of the patient's wrists and each ankles, the four lines would lie in the frontal plane. Similarly, if a straight line were drawn from the heart to each of the six electrodes placed on the patient's chest, the six lines would generally lie in the horizontal plane. FIG. 2 illustrates a cross-section of a patient's upper torso and the placement of the standard ECG electrodes in the horizontal plane. The electrical signals derived from the electrodes in the horizontal plane are referred to as the precordial leads and include leads V1, V2, V3, V4, V5, and V6.

The precordial leads are conventionally obtained with various permutations of the six electrodes attached to the patient's left chest, in addition to three of the four limb electrodes. Each of the six precordial leads is comprised of the signal between the potential at the particular electrode placed on the patient's left chest and the potential at Wilson's central terminal. As shown in FIG. 3, Wilson's central terminal is the average of the potentials at the eRA, eLA, and eLL electrodes. For example, lead V1 is the signal between electrode eV1 and Wilson's central terminal (WCT). Thus, in order to obtain one precordial lead of standard ECG data, at least three electrodes must be attached to the patient's limbs and at least one electrode must be attached to the patient's chest.

However, in some ECG applications it becomes inconvenient or impossible to place the electrodes on the patient's limbs, because the patient must be free to move. Applications where the patient must be free to move include long term recordings, such as Holter monitoring and event recording; ambulatory patient monitoring, such as telemetry monitoring; and exercise testing on treadmills or bicycles, known as stresstesting. In these tests, the limb electrode positions are unacceptable for electrode placement due to inconvenience, increased danger of tangling of the leadwires, and increased noise from limbs in motion.

### SUMMARY OF INVENTION

In light of the limitations described above, a need exists for a method and apparatus for generating precordial leads without attaching electrodes to the patient's limbs.

Accordingly, the invention provides a method and apparatus for generating a plurality of ECG precordial leads using a precordial central terminal derived from a plurality of electrodes for attachment around the circumference of a patient's upper torso.

The apparatus is a device for acquiring and processing electrical signals produced by a patient's heart. The device includes a plurality of electrodes for attachment to the patient's upper torso. In one embodiment, the plurality of electrodes includes at least one electrode for attachment to the patient's chest and at least one electrode for attachment to the patient's back. The device also includes an acquisition module coupled to the plurality of electrodes for acquiring electrical signals from the electrodes and a signal processor coupled to the acquisition module for generating a plurality of ECG precordial leads from the acquired electrical signals. The signal processor generates a plurality of ECG precordial leads from the acquired electrical signals by generating an approximation of an electrical potential near the center of the patient's heart (i.e., a precordial central terminal), and subtracting the approximation of the electrical potential near the center of the patient's heart from each of the signals acquired from the electrodes on the patient's chest.

The plurality of electrodes may be coupled to a belt adapted to be attached around the circumference of the patient's upper torso. The acquisition module and a transmitter may be coupled to the belt in order to transmit the acquired electrical signals to a remote location. A receiver may be coupled to the signal processor and wirelessly coupled to the transmitter for receiving the acquired electrical signals.

For the method of the invention, a plurality of electrodes are positioned on the patient's upper torso. The plurality of electrodes positioned on the patient's upper torso may include at least one electrode positionable on the patient's chest and at least one electrode positionable on the patient's back. Electrical signals are acquired from the plurality of electrodes, and a plurality of ECG precordial leads are generated from the acquired electrical signals. The plurality of ECG precordial leads are generated by approximating an electrical potential near the center of the patient's heart (i.e., the precordial central terminal), and by subtracting the approximation of the electrical potential near the center of the patient's heart from each of the signals acquired from the electrodes on the patient's chest.

The plurality of electrodes may be coupled to a belt and the belt may be wrapped around the patient's upper torso just slightly below the patient's breast so that each one of the plurality of electrodes is positioned in a plane generally perpendicular to a longitudinal axis approximately defined by the patient's spine.

Various other features and advantages of the invention are set forth in the following drawings, detailed description and claims.

### BRIEF DESCRIPTION OF DRAWINGS

FIG. 1 illustrates the electrode placement for a standard, twelve-lead ECG.

FIG. 2 illustrates a cross-section view of a patient's chest and the location of the precordial leads in the horizontal plane for a standard, twelve-lead ECG.

FIG. 3 illustrates the electrode placement and electrical connections for generating Wilson's central terminal and lead V1.

FIG. 4 illustrates the apparatus embodying the invention connected to a patient.

FIG. 5 illustrates a cross-section view of a patient's chest and the electrode placement according to one preferred embodiment of the invention.

FIG. 6 illustrates a view of a patient's chest and the electrode placement for the preferred embodiment of the invention of FIG. 5.

FIG. 7 illustrates a view of a patient's back and the electrode placement for the preferred embodiment of the invention of FIG. 5.

FIG. 8 illustrates a view of a patient's back and the electrode placement for another preferred embodiment of the invention.

FIG. 9 is a flow chart illustrating one preferred embodiment of the method of the invention.

FIG. 10 is a flow chart illustrating another preferred embodiment of the method of the invention.

### DETAILED DESCRIPTION

Before one embodiment of the invention is explained in full detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of including and comprising and variations thereof herein is meant to encompass the items listed thereafter and equivalents thereof as well as additional items.

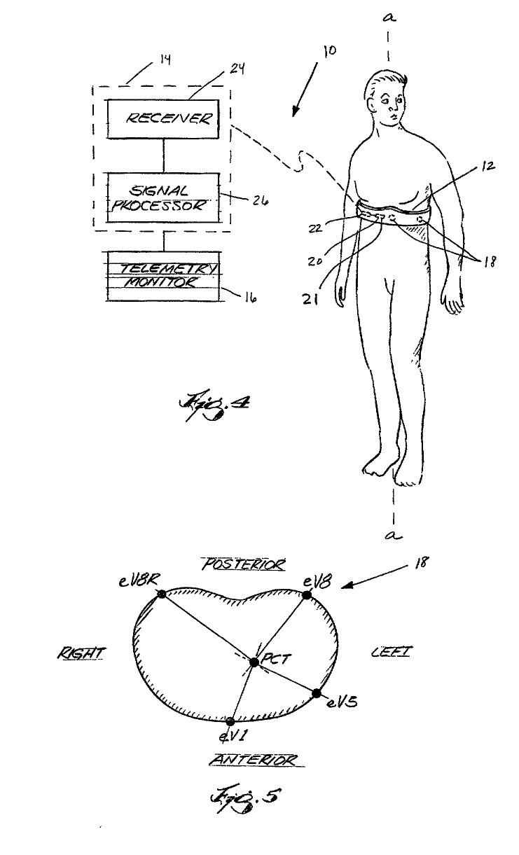

FIG. 4 illustrates an ECG device 10 embodying the invention. The ECG device 10 includes a belt 12 attached to a patient's upper torso. The belt 12 includes electrodes 18, an acquisition module 20, and a transmitter 22. In one preferred embodiment of the invention, the belt 12 is a Polar® coded transmitter belt constructed of a rubber material. However, the belt 12 may be any type of belt having a compact, lightweight, and ergonomic design and having a length suitable for attachment to the patient's upper torso slightly below the patient's breast. The transmitter 22 is remotely coupled to an ECG module 14 and to a telemetry monitor 16 coupled to the ECG module 14. While the invention is described in the context of telemetry monitoring, the use of other devices for acquiring ECG signals, such as, for example, a bedside monitor, transport monitor, Holter monitor, or event recorder, is contemplated by the invention.

The electrodes 18 are coupled to the belt 12 so that when the belt 12 is attached to the circumference of the patient's upper torso, each one of the electrodes 18 is generally positioned in a single plane perpendicular to a longitudinal axis a-a approximately defined by the patient's spine. Any number of electrodes 18 may be coupled to the belt 12, as long as the electrodes 18 are positioned around the circumference of the patient's upper torso when the belt 12 is attached to the patient. The electrodes 18 are coupled to the belt 12 so that when the belt 12 is attached to the patient's upper torso, a contact surface (not shown) of each one of the electrodes 18 properly contacts the patient's skin in order to acquire electrical signals generated by the patient's heart while minimizing interference. The electrodes 18 may be secured to the belt 12 in any conventional manner, such as by being integrally molded, sewn, glued, riveted, snapped, or otherwise fastened to the belt 12. Moreover, although the electrodes 18 are described and shown in the drawings as being coupled to the belt 12, the electrodes 18 may be attached to the patient's upper torso using individual leadwires in a conventional manner without being coupled to the belt 12.

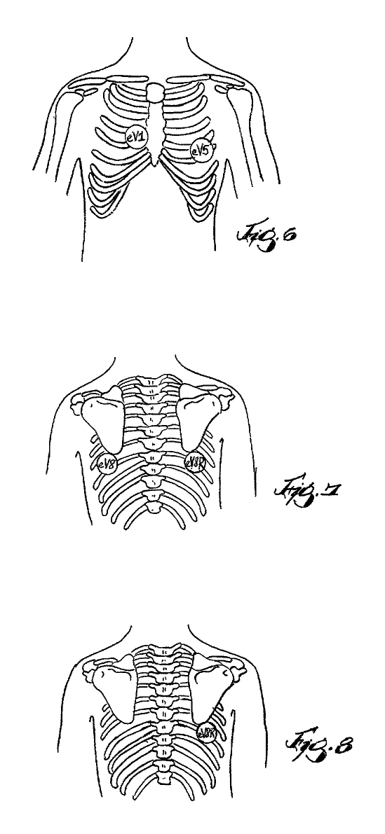

In one preferred embodiment of the invention, as shown in FIGS. 5, 6, and 7, four electrodes are positioned on the patient's upper torso. Referring to FIGS. 5 and 6, the electrodes 18 include a first electrode (eV1) to be positioned on the patient's chest in approximately the fourth intercostal space at the right border of the patient's sternum and a second electrode (eV5) to be positioned on the patient's chest in approximately the fifth intercostal space at the anterior axillary line. If the eV1 and eV5 electrodes are positioned on the patient's upper torso via the belt 12, the eV1 electrode may be positioned slightly lower and the eV5 electrode may be positioned slightly higher than their standard ECG electrode positions. Referring to FIGS. 5 and 7, the electrodes 18 include a third electrode (eV8) to be positioned on the patient's back in approximately the fifth intercostal space under the left mid-scapular line and a fourth electrode (eV8R) to be positioned on the patient's back in approximately the fifth intercostal space under the right mid-scapular line.

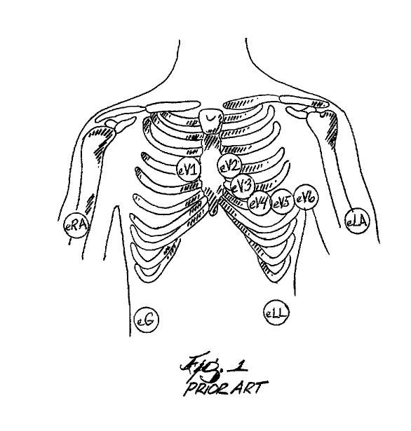

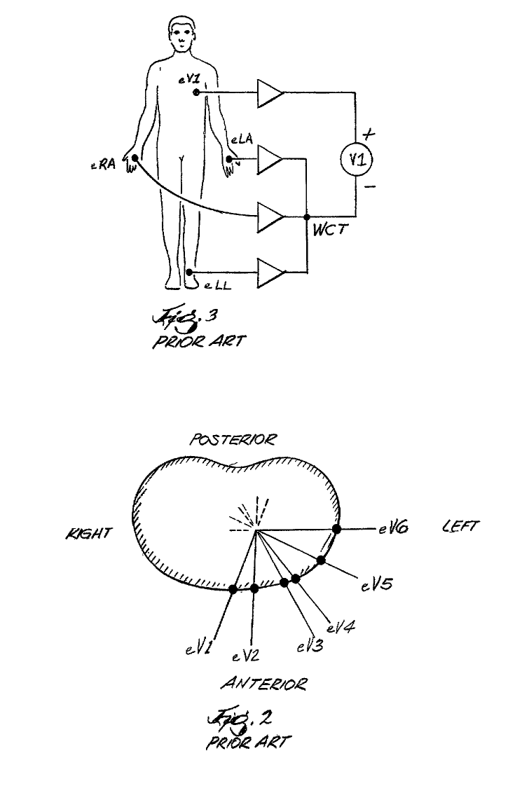

In another preferred embodiment of the invention, one electrode is positioned on the patient's back and one or more electrodes are positioned on the patient's chest. Referring to FIGS. 1 and 8, the electrodes 18 include an electrode (eV8R) to be positioned on the patient's back in approximately the fifth intercostal space under the right mid-scapular line and one or more electrodes (e.g., eV1, eV2, eV3, eV4, eV5, and eV6) to be positioned on the patient's chest.

Referring to FIG. 4, the acquisition module 20 is electrically coupled to each one of the electrodes 18. As shown in FIG. 4, the acquisition module 20 is coupled to the belt 12. In other embodiments (not shown), the acquisition module 20 may be contained within a separate housing (not shown), such as that of a Holter monitor or an event recorder, and may be electrically coupled to the belt 12. For example, the belt 12 can be positioned on the patient's upper torso under the patient's clothing and the Holter monitor or event recorder can be attached over the patient's clothing (e.g., to the patient's pants waistband or belt) at the patient's waist and coupled to the belt 12 via a conventional cable. The acquisition module 20 is used to acquire, process, and temporarily store electrical signals from the electrodes 18.

The acquisition module 20 includes a signal processor 21. In one preferred embodiment, the signal processor 21 is an analog signal processor comprised of a resistor network coupled to an operational amplifier. In another preferred embodiment, the signal processor 21 is a digital signal processor including four amplifiers and a four-channel analog-to-digital converter coupled to a microprocessor within the acquisition module 20. The signal processor 21 generates an approximation of the electrical potential near the center of the patient's heart based on the electrical signals acquired from the plurality of electrodes 18. The approximation of the electrical potential near the center of the patient's heart generated from the signals received from the electrodes 18 is referred to as the precordial central terminal (labeled PCT in FIG. 5). The precordial central terminal is then used by the signal processor 21 as a reference electrode, in lieu of Wilson's central terminal, to generate the precordial leads.

The acquisition module 20 may include any conventional type of memory, such as RAM memory, for temporarily storing the generated precordial leads. The acquisition module 20 may include enough memory to store the precordial leads for an extended period of time (e.g., one to two days for a Holter monitor or up to 30 days for an event recorder), or only enough memory to temporarily store the precordial leads before the precordial leads are transmitted to a remote location.

In order to transmit the precordial leads to a remote location, a conventional wireless transmitter 22 is coupled to the acquisition module 20. While the transmitter 22 is shown in FIG. 4 as being coupled to the belt 12, the transmitter 22 may alternatively be contained within the separate housing of a Holter monitor or an event recorder. The transmitter 22 is capable of transmitting RF, infrared, microwave, or any other conventional frequency signals used for wireless communications. The signals generated by transmitter 22 are received by a conventional wireless receiver 24 and may be further processed by a signal processor 26 within the ECG module 14.

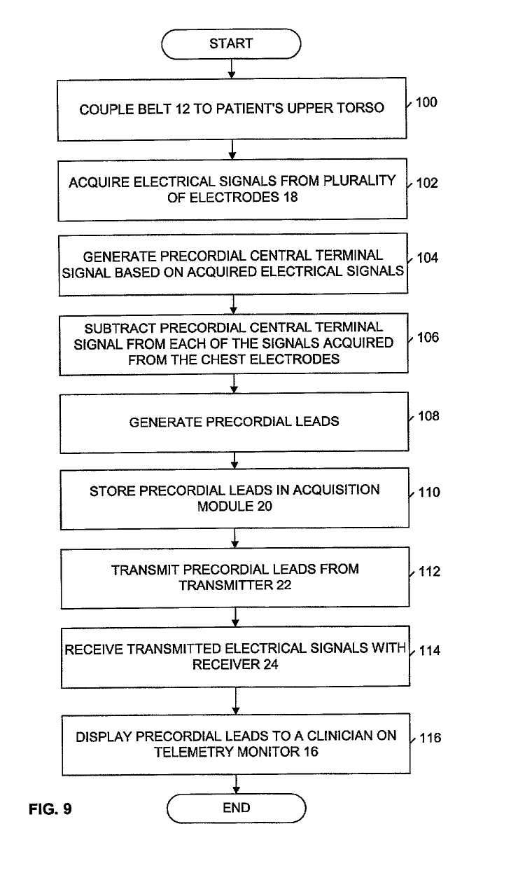

Referring to FIGS. 4 and 9, in operation, the belt 12 is coupled 100 to the patient's upper torso slightly below the patient's breast so that each one of the plurality of electrodes 18 is generally positioned in a single plane perpendicular to a longitudinal axis a-a approximately defined by the patient's spine. Electrical signals generated by the patient's heart are acquired 102 from the electrodes 18.

The signal processor 21 generates 104 the precordial central terminal based on the acquired electrical signals received by receiver 24. For the electrode placement of the preferred embodiment shown in FIG. 5, in order to generate the precordial central terminal signal, the signal processor 21 performs a weighted combination of the signals acquired from the eV1, eV5, eV8, and eV8R electrodes according to the following equation: PCT=A(eV1)+B(eV5)+C(eV8)+D(eV8R) where PCT is the precordial central terminal signal, A is a first weighted coefficient, eV1 is the signal acquired from the eV1 electrode, B is a second weighted coefficient, eV5 is the signal acquired from the eV5 electrode, C is a third weighted coefficient, eV8 is the signal acquired from the eV8 electrode, D is a fourth weighted coefficient, and eV8R is the signal acquired from the eV8R electrode. The A, B, C, and D weighted coefficients are the appropriate coefficients to implement the weighted combination of the electrical signals acquired from the electrodes 18.

For the electrode placement of the preferred embodiment shown in FIG. 8, the signal processor 21 uses the signal acquired from the eV8R electrode as the precordial central terminal signal. The position of the eV8R electrode (on the patient's back under the patient's right shoulder blade) is as far from the patient's heart as an electrode can be placed while still being generally within the single plane perpendicular to the longitudinal axis a-a defined by the patient's spine. The signal acquired from the eV8R electrode serves as a far field reference from the patient's heart, and thus, can serve as an approximation of Wilson's central terminal, i.e., the precordial central terminal.

In both of the preferred embodiments, the precordial central terminal is an approximation of Wilson's central terminal, but the precordial central terminal is generated only from electrodes attached to the patient's upper torso in the horizontal plane, rather than from electrodes attached to the patient's limbs in the frontal plane.

Referring to FIGS. 4 and 9, once the precordial central terminal signal is generated 104, the precordial central terminal signal is subtracted 106 from each one of the signals acquired from the electrodes 18 positioned on the patient's chest in order to generate 108 the precordial leads. For example, precordial leads V1 and V5 are generated according to the following equations: V1=eV1 PCTwhere V1 is the V1 precordial lead, eV1 is the signal acquired from the eV1 electrode, and PCT is the precordial central terminal signal; and V5=eV5 PCT where V5 is the V5 precordial lead, eV5 is the signal acquired from the eV5 electrode, and PCT is the precordial central terminal signal.

The generated precordial leads are temporarily stored 110 in the acquisition module 20. The precordial leads are then transmitted 112 from the transmitter 22 and received 114 by the receiver 24. The precordial leads may be further processed by a signal processor 26 and are then displayed 116 to a clinician on the telemetry monitor 16.

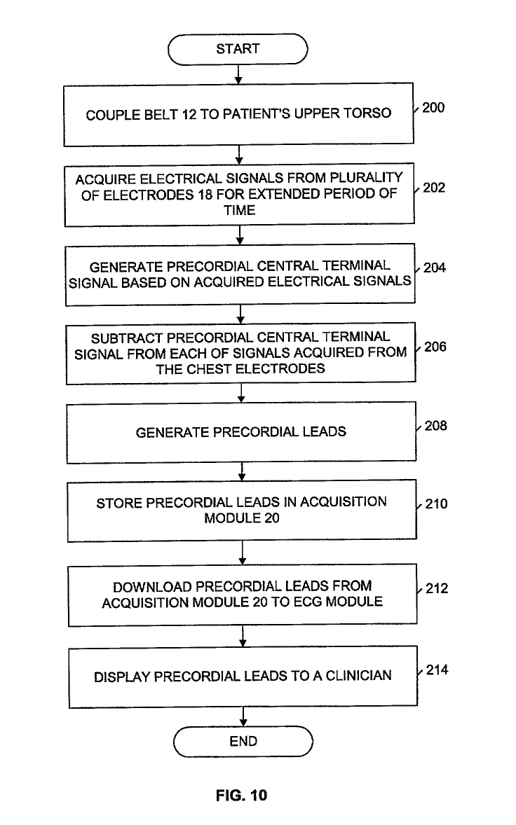

According to another preferred method of the invention, electrical signals are acquired, processed, and stored in a device such as a Holter monitor or an event recorder. Referring to FIGS. 4 and 10, the belt 12 is coupled 200 to the patient's upper torso slightly below the patient's breast so that each one of the electrodes 18 is generally positioned in a single plane perpendicular to a longitudinal axis a-a approximately defined by the patient's spine. Electrical signals generated by the patient's heart are acquired 202 via the electrodes 18 for an extended period of time. For example, if the acquisition device 20 is a Holter monitor, electrical signals are acquired from the plurality of electrodes for one to two days. If the acquisition device 20 is an event recorder, electrical signals are acquired from the plurality of electrodes for up to 30 days. Similar to the method described above with respect to the telemetry-based device, the signal processor 21 then generates 204 the precordial central terminal signal based on the acquired signals. The precordial central terminal signal is subtracted 206 from each one of the signals acquired from the electrodes positioned on the patient's chest in order to generate 208 the precordial leads. The acquired electrical signals are stored 210 in the memory of the acquisition module 20.

Once the electrical signals have been acquired for the extended period of time and once the precordial leads have been generated, the precordial leads are downloaded 212 from the acquisition module 20 to the ECG module 14. The precordial leads may be further processed by the signal processor 26 and are then displayed 214 to a clinician.

Various features and advantages of the invention are set forth in the following claims.

What is claimed

1. A device for acquiring and processing electrical signals produced by a patient's heart, the device comprising:
a plurality of electrodes for attachment to the patient's upper torso, wherein the plurality of electrodes does not include electrodes for attachment to the patient's limbs;
an acquisition module coupled to the plurality of electrodes for acquiring electrical signals from the plurality of electrodes; and
a signal processor coupled to the acquisition module for generating a plurality of electrocardiogram precordial leads from the acquired signals.

2. The device of claim 1 and further comprising a belt adapted to be attached around the circumference of the patient's upper torso, and wherein the plurality of electrodes are coupled to the belt so that when the belt is attached to the patient each one of the plurality of electrodes is generally positioned in a plane perpendicular to a longitudinal axis approximately defined by the patient's spine.

3. The device of claim 2 wherein the belt is adapted to be attached around the circumference of the patient's upper torso at a level slightly below the patient's breast.

4. The device of claim 2 and further comprising a transmitter coupled to the acquisition module and a receiver coupled to an electrocardiogram machine, wherein the transmitter, the acquisition module, and the signal processor are coupled to the belt, wherein the receiver is coupled to the electrocardiogram machine, and wherein the plurality of electrocardiogram precordial leads are wirelessly transmitted from the transmitter to the receiver.

5. The device of claim 1 wherein the signal processor generates a plurality of electrocardiogram precordial leads from the acquired electrical signals by generating an approximation of an electrical potential near the center of the patient's heart based on the acquired electrical signals.

6. The device of claim 5 wherein the approximation of the electrical potential near the center of the patient's heart is an approximation of Wilson's central terminal.

7. The device of claim 5 wherein the signal processor generates an approximation of the electrical potential near the center of the patient's heart by determining a weighted combination of a plurality of the acquired electrical signals.

8. The device of claim 1 wherein the plurality of electrodes includes a first electrode attachable to the patient's chest in approximately the fourth intercostal space at the right border of the sternum, a second electrode attachable to the patient's chest in approximately the fifth intercostal space at the anterior axillary line, a third electrode attachable to the patient's back in approximately the fifth intercostal space under the left mid-scapular line, and a fourth electrode attachable to the patient's back in approximately the fifth intercostal space under the right mid-scapular line.

9. The device of claim 8 wherein the signal processor generates an approximation of the electrical potential near the center of the patient's heart by determining a weighted combination of the signals acquired from the plurality of electrodes.

10. The device of claim 9 wherein the signal processor generates each one of the plurality of electrocardiogram precordial leads by subtracting the approximation of the electrical potential near the center of the patient's heart from each one of the signals acquired from the first electrode and the second electrode.

11. The device of claim 1 wherein the plurality of electrodes includes a first electrode attachable to the patient's back in approximately the fifth intercostal space under the right mid-scapular line and at least one electrode attachable to the patient's chest.

12. The device of claim 11 wherein the signal processor uses the signal acquired from the first electrode as an approximation of the electrical potential near the center of the patient's heart.

13. The device of claim 12 wherein the signal processor generates each one of the plurality of electrocardiogram precordial leads by subtracting the approximation of the electrical potential near the center of the patient's heart from each one of the signals acquired from the at least one electrode on the patient's chest.

14. The device of claim 1 wherein the acquisition module is capable of storing precordial leads for approximately one month.

15. The device of claim 1 and further comprising an electrocardiogram machine wirelessly coupled to the acquisition module and a telemetry monitor coupled to the electrocardiogram machine.

16. An electrocardiogram device for acquiring and processing electrical signals produced by a patient's heart, the device comprising:
a belt adapted to be attached to the patient's upper torso;
a plurality of electrodes coupled to the belt, wherein the plurality of electrodes does not include electrodes for attachment to the patient's limbs;
an acquisition module coupled to the belt and the plurality of electrodes for acquiring electrical signals from the plurality of electrodes;
a signal processor coupled to the acquisition module for generating a plurality of electrocardiogram precordial leads from the acquired electrical signals;
a transmitter coupled to the acquisition module for transmitting the plurality of electrocardiogram precordial leads; and
a receiver wirelessly coupled to the transmitter for receiving the acquired electrical signals.

17. The device of claim 16 wherein the plurality of electrodes are coupled to the belt so that when the belt is attached to the patient each one of the plurality of electrodes is generally positioned in a plane perpendicular to a longitudinal axis approximately defined by the patient's spine.

18. The device of claim 17 wherein the belt is adapted to be attached around the circumference of the patient's upper torso at a level slightly below the patient's breast.

19. The device of claim 16 wherein the signal processor generates a plurality of electrocardiogram precordial leads from the acquired electrical signals by generating an approximation of an electrical potential near the center of the patient's heart based on the acquired electrical signals.

20. The device of claim 19 wherein the approximation of the electrical potential near the center of the patient's heart is an approximation of Wilson's central terminal.

21. The device of claim 19 wherein the signal processor generates an approximation of the electrical potential near the center of the patient's heart by determining a weighted combination of a plurality of the acquired electrical signals.

22. The device of claim 16 wherein the plurality of electrodes includes a first electrode positionable on the patient's chest in approximately the fourth intercostal space at the right border of the sternum, a second electrode positionable on the patient's chest in approximately the fifth intercostal space at the anterior axillary line, a third electrode positionable on the patient's back in approximately the fifth intercostal space under the left mid-scapular line, and a fourth electrode positionable on the patient's back in approximately the fifth intercostal space under the right mid-scapular line.

23. The device of claim 22 wherein the signal processor generates an approximation of the electrical potential near the center of the patient's heart by determining a weighted combination of the signals acquired from the plurality of electrodes.

24. The device of claim 23 wherein the signal processor generates each one of the plurality of electrocardiogram precordial leads by subtracting the approximation of the electrical potential near the center of the patient's heart from each one of the signals acquired from the first electrode and the second electrode.

25. The device of claim 16 wherein the plurality of electrodes includes a first electrode positionable on the patient's back in approximately the fifth intercostal space under the right mid-scapular line and at least one electrode positionable on the patient's chest.

26. The device of claim 25 wherein the signal processor uses the signal acquired from the first electrode as an approximation of the electrical potential near the center of the patient's heart.

27. The device of claim 26 wherein the signal processor generates each one of the plurality of electrocardiogram precordial leads by subtracting the approximation of the electrical potential near the center of the patient's heart from each one of the signals acquired from the at least one electrode on the patient's chest.

28. The device of claim 16 wherein the acquisition module is capable of storing precordial leads for approximately one month.

29. The device of claim 16 and further comprising an electrocardiogram machine wirelessly coupled to the acquisition module and a telemetry monitor coupled to the electrocardiogram machine.

30. An acquisition device for attachment to a patient and for acquiring electrical signals produced by the patient's heart, the device comprising:
a belt adapted to be attached to the patient's upper torso;
a plurality of electrodes coupled to the belt, the plurality of electrodes including at least one electrode positioned within the belt so that when the belt is attached to the patient the electrode contacts the patient's chest, and at least one electrode positioned within the belt so that when the belt is attached to the patient the electrode contacts the patient's back, wherein the plurality of electrodes does not include electrodes for attachment to the patient's limbs;
an acquisition module including a signal processor coupled to the belt and the plurality of electrodes for acquiring electrical signals from the plurality of electrodes and for generating a plurality of electrocardiogram precordial leads from the acquired signals; and
a transmitter coupled to the acquisition module for transmitting the plurality of electrocardiogram precordial leads to a remote location.

31. The device of claim 30 wherein the plurality of electrodes are coupled to the belt so that when the belt is attached to the patient each one of the plurality of electrodes is generally positioned in a plane perpendicular to a longitudinal axis approximately defined by the patient's spine.

32. The device of claim 30 wherein the belt is attachable around the circumference of the patient's upper torso at a level slightly below the patient's breast.

33. The device of claim 30 and further comprising a receiver located at the remote location, wherein the acquired signals are wirelessly transmitted from the transmitter to the receiver.

34. The device of claim 30 wherein the signal processor generates a plurality of electrocardiogram precordial leads from the acquired electrical signals by generating an approximation of an electrical potential near the center of the patient's heart based on the acquired electrical signals.

35. The device of claim 34 wherein the approximation of the electrical potential near the center of the patient's heart is an approximation of Wilson's central terminal.

36. The device of claim 34 wherein the signal processor generates an approximation of the electrical potential near the center of the patient's heart by determining a weighted combination of a plurality of the acquired electrical signals.

37. The device of claim 30 wherein the plurality of electrodes includes a first electrode positionable on the patient's chest in approximately the fourth intercostal space at the right border of the sternum, a second electrode positionable on the patient's chest in approximately the fifth intercostal space at the anterior axillary line, a third electrode positionable on the patient's back in approximately the fifth intercostal space under the left mid-scapular line, and a fourth electrode positionable on the patient's back in approximately the fifth intercostal space under the right mid-scapular line.

38. The device of claim 37 wherein the signal processor generates an approximation of the electrical potential near the center of the patient's heart by determining a weighted combination of the signals acquired from the plurality of electrodes.

39. The device of claim 38 wherein the signal processor generates each one of the plurality of electrocardiogram precordial leads by subtracting the approximation of the electrical potential near the center of the patient's heart from each one of the signals acquired from the first electrode and the second electrode.

40. The device of claim 30 wherein the plurality of electrodes includes a first electrode positionable on the patient's back in approximately the fifth intercostal space under the right mid-scapular line and at least one electrode positionable on the patient's chest.

41. The device of claim 40 wherein the signal processor uses the signal acquired from the first electrode as an approximation of the electrical potential near the center of the patient's heart.

42. The device of claim 41 wherein the signal processor generates each one of the plurality of electrocardiogram precordial leads by subtracting the approximation of the electrical potential near the center of the patient's heart from each one of the signals acquired from the at least one electrode on the patient's chest.

43. The device of claim 30 wherein the acquisition module is capable of storing precordial leads for approximately one month.

44. The device of claim 30 and further comprising an electrocardiogram machine wirelessly coupled to the acquisition module and a telemetry monitor coupled to the electrocardiogram machine.

45. A method of acquiring and processing electrical signals produced by a patient's heart, the method comprising the acts of:
positioning a plurality of electrodes on the patient's upper torso, without positioning electrodes on the patient's limbs;
acquiring electrical signals from the plurality of electrodes; and
generating a plurality of electrocardiogram precordial leads from the acquired electrical signals.

46. The method of claim 45 wherein the plurality of electrodes are coupled to a belt and wherein the act of positioning a plurality of electrodes on the patient's upper torso includes the act of wrapping the belt around the circumference of the patient's upper torso so that each one of the plurality of electrodes is generally positioned in a plane perpendicular to a longitudinal axis approximately defined by the patient's spine.

47. The method of claim 46 wherein the act of wrapping a belt around circumference of the patient's upper torso includes the act of wrapping a belt around the circumference of the patient's upper torso at a level slightly below the patient's breast.

48. The method of claim 46 and further comprising the act of wirelessly transmitting the plurality of electrocardiogram precordial leads from a transmitter coupled to the belt to a receiver located in a remote location.

49. The method of claim 45 wherein the act of generating a plurality of electrocardiogram precordial leads from the acquired electrical signals includes the act of generating an approximation of an electrical potential near the center of the patient's heart based on the acquired electrical signals.

50. The method of claim 49 wherein the act of generating an approximation of an electrical potential near the center of the patient's heart based on the acquired electrical signals includes the act of generating an approximation of Wilson's central terminal.

51. The method of claim 49 wherein the act of generating an approximation of the electrical potential near the center of the patient's heart includes the act of determining a weighted combination of a plurality of the acquired electrical signals.

52. The method of claim 45 wherein the act of positioning a plurality of electrodes on the patient's upper torso includes the act of positioning a first electrode on the patient's chest in approximately the fourth intercostal space at the right border of the sternum, a second electrode on the patient's chest in approximately the fifth intercostal space at the anterior axillary line, a third electrode on the patient's back in approximately the fifth intercostal space under the left mid-scapular line, and a fourth electrode on the patient's back in approximately the fifth intercostal space under the right mid-scapular line.

53. The method of claim 52 wherein the act of generating an approximation of the electrical potential near the center of the patient's heart includes the act of determining a weighted combination of the signals acquired from the plurality of electrodes.

54. The method of claim 53 wherein the act of generating a plurality of electrocardiogram precordial leads from the acquired electrical signals includes the act of generating each one of the plurality of electrocardiogram precordial leads by subtracting the approximation of the electrical potential near the center of the patient's heart from each one of the signals acquired from the first electrode and the second electrode.

55. The method of claim 45 wherein the act of positioning a plurality of electrodes on the patient's upper torso includes the act of positioning a first electrode in approximately the fifth intercostal space under the right mid-scapular line and at least one electrode on the patient's chest.

56. The method of claim 55 wherein the act of generating an approximation of the electrical potential near the center of the patient's heart includes the act of using the signal acquired from the first electrode as an approximation of the electrical potential near the center of the patient's heart.

57. The method of claim 56 wherein the act of generating a plurality of electrocardiogram precordial leads from the acquired electrical signals includes the act of generating each one of the plurality of electrocardiogram precordial leads by subtracting the approximation of the electrical potential near the center of the patient's heart from each one of the signals acquired from the at least one electrode on the patient's chest.

58. The method of claim 45 wherein the act of acquiring electrical signals from the plurality of electrodes includes the act of acquiring electrical signals for approximately one month.

59. The method of claim 45 and further comprising the act of wirelessly coupling a telemetry monitor to the plurality of electrodes.

60. A method of acquiring and processing electrical signals produced by a patient's heart, the method comprising the acts of:
positioning a plurality of electrodes on the patient's upper torso, the plurality of electrodes including at least one electrode positionable on the patient's chest and at least one electrode positionable on the patient's back, wherein the plurality of electrodes does not include electrodes for positioning on the patient's limbs;
acquiring electrical signals from the plurality of electrodes;
generating an approximation of an electrical potential near the center of the patient's heart by determining a weighted combination of a plurality of the acquired electrical signals; and
generating a plurality of electrocardiogram precordial leads from the acquired electrical signals by subtracting the approximation of the electrical potential near the center of the patient's heart from each one of the signals acquired from the at least one electrode on the patient's chest.

61. The method of claim 60 wherein the plurality of electrodes are coupled to a belt and wherein the act of positioning a plurality of electrodes on the patient's upper torso includes the act of wrapping the belt around the circumference of the patient's upper torso so that each one of the plurality of electrodes is generally positioned in a plane perpendicular to a longitudinal axis approximately defined by the patient's spine.

62. The method of claim 61 wherein the act of wrapping a belt around the circumference of the patient's upper torso includes the act of wrapping a belt around the circumference of the patient's upper torso at a level slightly below the patient's breast.

63. The method of claim 61 and further comprising the act of wirelessly transmitting the plurality of electrocardiogram precordial leads from a transmitter coupled to the belt to a receiver.

64. The method of claim 60 wherein the act of generating an approximation of an electrical potential near the center of the patient's heart based on the acquired electrical signals includes the act of generating an approximation of Wilson's central terminal.

65. The method of claim 60 wherein the act of positioning a plurality of electrodes on the patient's upper torso includes the act of positioning a first electrode on the patient's chest in approximately the fourth intercostal space at the right border of the sternum, a second electrode on the patient's chest in approximately the fifth intercostal space at the anterior axillary line, a third electrode on the patient's back in approximately the fifth intercostal space under the left mid-scapular line, and a fourth electrode on the patient's back in approximately the fifth intercostal space under the right mid-scapular line.

66. The method of claim 65 wherein the act of generating an approximation of the electrical potential near the center of the patient's heart includes the act of determining a weighted combination of the signals acquired from the plurality of electrodes.

67. The method of claim 66 wherein the act of generating a plurality of electrocardiogram precordial leads from the acquired electrical signals includes the act of generating each one of the plurality of electrocardiogram precordial leads by subtracting the approximation of the electrical potential near the center of the patient's heart from each one of the signals acquired from the first electrode and the second electrode.

68. The method of claim 60 wherein the act of positioning a plurality of electrodes on the patient's upper torso includes the act of positioning a first electrode in approximately the fifth intercostal space under the right mid-scapular line and at least one electrode on the patient's chest.

69. The method of claim 68 wherein the act of generating an approximation of the electrical potential near the center of the patient's heart includes the act of using the signal acquired from the first electrode as an approximation of the electrical potential near the center of the patient's heart.

70. The method of claim 69 wherein the act of generating a plurality of electrocardiogram precordial leads from the acquired electrical signals includes the act of generating each one of the plurality of electrocardiogram precordial leads by subtracting the approximation of the electrical potential near the center of the patient's heart from each one of the signals acquired from the at least one electrode on the patient's chest.

71. The method of claim 60 wherein the act of acquiring electrical signals from the plurality of electrodes includes the act of acquiring electrical signals for approximately one month.

72. The method of claim 60 and further comprising the act of wirelessly coupling a telemetry monitor to the plurality of electrodes.
